(19) United States
(12) Patent Application Publication
(10) Pub. No.: US 2020/0129117 A1
(43) Pub. Date: Apr. 30, 2020

(54) Condition Detector

(71) Applicant: Altair Medical Ltd., Motherwell (GB)

(72) Inventor: Bruce Colin Henderson, Motherwell (GB)

(21) Appl. No.: 16/396,449

(22) Filed: Apr. 26, 2019

(30) Foreign Application Priority Data
Oct. 29, 2018 (GB) 1817649.5

(51) Int. Cl. A61B 5/00; A61B 5/11; A61B 5/113; A61B 5/08; A61B 5/16

(52) U.S. Cl. CPC A61B 5/4833; A61B 2562/0219; A61B 5/1135; A61B 5/0816; A61B 5/4839; A61B 5/6803; A61B 5/6843; A61B 5/0002; A61B 5/7235; A61B 5/7207; A61B 5/7246; A61B 5/16; A61B 5/746; A61B 5/6833; A61B 5/7275; A61B 5/1114

(57) ABSTRACT

An overdose detector that includes a motion sensor mounted or mountable on a chest wall and configured to detect chest wall movement, and wherein the overdose detector is configured to detect and/or predict an overdose based on the detected chest wall movement. Also described herein are associated systems, methods and computer program products.

### FIELD

Described herein is a condition detector, such as an overdose detector, which, for example, can be used to detect opioid overdoses. The present disclosure also relates to an associated method of using a condition detector.

### BACKGROUND

Opioids can be prescribed to treat pain. However, when using opioids there is a risk of overdose, which can result in death. Antidotes are available to treat opioid overdoses, but the antidote must be administered quickly to prevent death.

It is at least one objective of at least one embodiment of the present disclosure to provide an improved overdose detector.

### SUMMARY

Aspects of the disclosure are defined by the independent claims appended herewith. Preferred features are defined by the dependent claims appended herewith.

According to a first aspect of the present disclosure there is provided a condition detector, which may be a drug dose compliance detector, such as an opioid dose-compliance detector. The drug dose compliance detector may be an overdose detector and the condition detected or predicted by the detector may be or comprise an overdose. The condition detected or predicted by the detector may be or comprise a loss of consciousness, such as a loss of consciousness due to the overdose, e.g. opioid overdose. The condition detected or predicted by the detector may comprise chronic obstructive pulmonary disease (COPD), asthma, heart failure or disfunction, and/or the like. The detector may be wearable, e.g. configured to be worn by a wearer. The detector may be configured for mounting to a human chest, upper abdomen or upper torso, such as directly mounting to a human chest, upper abdomen or upper torso.

The detector may comprise a motion sensor, such as an accelerometer. The motion sensor may be wearable, e.g. configured to be worn by the wearer. The motion sensor may be a chest mountable detector. The motion sensor may be configured for mounting to a human chest, upper abdomen or upper torso. The motion sensor may be configured to determine chest wall movement. The detector may be configured to detect and/or predict the condition, e.g. an overdose, non-compliance with a set dose or loss of consciousness, of a wearer of the detector from the detected chest wall movement detected by the motion sensor.

The motion sensor may be configured to produce an output signal that is representative of chest wall movement, for example the output signal may be indicative of a position, a velocity and/or an acceleration of the chest wall. The motion sensor may be configured to monitor the movement of the chest wall over time, such as the position, velocity and/or acceleration of the chest wall over time. The motion sensor may be configured to determine turning points, transitions or points of inflexion in the movement of the chest wall, which may comprise, for example, detecting transitions between inhalation and exhalation and/or vice-versa, which may comprise temporary periods where the chest wall velocity is zero.

The detector may comprise and/or be configured to communicate with a processing system. The processing system may be entirely comprised in the detector or at one or more local or remote locations or distributed, e.g. functions of the processing system may be provided in the detector, at the local location and/or at the remote location. For example, the processing system may comprise at least one of: a detector processing module comprised in the detector; a local processing system that may be at the local location; and/or a remote processing system that may be at the remote location. For example, the local processing system may comprise or be comprised in a digital user device such as a smartphone, personal computer, laptop, tablet computer, phablet, PDA, or other smart device. The remote processing system may comprise or be comprised in a computer, a server system, a workstation, and/or the like.

The detector may comprise a communication system. The communication system may be configured to communicate with the local processing system and/or the remote processing system. The communication system may be configured to wirelessly communicate with the local and/or remote processing system. The communication system may be configured to communicate using Bluetooth, ZigBee, WiMAX, Li-Fi, near field communications (NFC) and/or WiFi. The communication system may be configured to communicate with the remote processing system via the local processing system. The communication system may be configured to communicate with the local processing system via short or shorter range communications, e.g. via Bluetooth, ZigBee, Wi-Fi, Li-Fi, WiMAX, NFC, and/or the like. The communication system may be configured to communicate with the remote processing system via a network, e.g. a wide area network which may comprise long range communication methods such as LoRa WAN, cellular phone network, over the internet and/or using or via a suitable file or data transfer protocol (e.g. FTP).

The processing system may be configured to determine a breathing rate and/or breathing waveform. The processing system may be configured to use the determined chest wall movement to determine the breathing rate and/or breathing waveform. The processing system may be configured to use the determined breathing rate and/or breathing waveform to detect and/or predict an overdose, non-compliance with a set dose or loss of consciousness.

The processing system may be configured to analyse the determined breathing waveform to determine a parameter or change in parameter of the breathing waveform. The processing system may be configured to apply filtering to the determined breathing waveform, e.g. high band pass filtering and/or low band pass filtering. By filtering out determined motions that have a frequency higher than an upper threshold and/or lower than a lower threshold, then it may be possible to more reliably determine motion due to breathing from “noise” motions, such as those due to gravity, walking, travel, speech, and/or the like. This may improve the accuracy and/or viability of the detector.

The processing system may be configured to use the parameter or change in parameter of the breathing waveform to detect and/or predict an overdose, non-compliance with a set dose or loss of consciousness. The parameter of the breathing waveform may be or comprise maxima and/or minima in the breathing waveform, which may be representative of the turning points, transitions or points of inflexion in the movement of the chest wall, such as the transitions between inhalation and exhalation and/or vice-versa.

The parameter of the breathing waveform may comprise a breathing rate, amplitude, and/or shape of at least part or all of the breathing waveform. The change in parameter of the breathing waveform may comprise a change over time.

The processing system may be configured to determine a signature pattern in the breathing waveform. The processing system may be configured to match the determined signature pattern with a reference signature pattern. The reference signature patterns may be stored in a look up table or database, which may be accessible by the processing system. The processing system may be configured to detect changes in the signature pattern in the breathing waveform, such as a change in amplitude, rate and/or shape of the signature pattern with time. The processing system may be configured to detect and/or predict an overdose, non-compliance with a set dose or loss of consciousness from the signature pattern and/or from changes in the signature pattern detected in the breathing waveform and/or from the matching of the determined signature pattern with the reference signature pattern.

The changes in the signature pattern over time may be indicative of apnoea, i.e. an absence of breathing. The changes in the signature pattern may comprise an absence of a waveform associated with breathing, e.g. a determination that the breathing rate is zero.

The processing system may be configured to communicate with data storage, such as a hard drive or flash drive. The data storage may be comprised in the detector, in the digital user device and/or in the remote processing system. The data storage may store an algorithm. The processing system may be configured to run the algorithm.

The algorithm may be configured to one or more of:

use the determined chest wall movement to determine a breathing rate and/or breathing waveform;

use the determined breathing rate or breathing waveform to detect and/or predict an overdose, non-compliance with a set dose or loss of consciousness;

analyse the determined breathing waveform to determine a parameter or change in parameter of the breathing waveform; and

use the parameter or change in parameter of the breathing waveform to detect and/or predict an overdose, non-compliance with a set dose or loss of consciousness;

detect the signature pattern in the breathing waveform;

match the determined signature pattern with a reference signature pattern;

detect changes in the signature pattern in the breathing waveform, such as a change in amplitude, rate and/or shape of the signature pattern with time; and/or

detect and/or predict an overdose, non-compliance with a set dose or loss of consciousness from the signature pattern and/or from changes in the signature pattern detected in the breathing waveform and/or from the matching of the determined signature pattern with the reference signature pattern.

The algorithm may comprise or be comprised in an artificial intelligence “AI” or machine learning “ML” algorithm. The AI or ML algorithm may be at least partly trained on a known or generated training data set and/or on historical or previous data collected by the detector, which may be data for the wearer. The AI or machine learning algorithm may be configured to receive as inputs one or more of: breathing rate; the breathing waveform; parameters of the breathing waveform; the signature pattern in the breathing waveform; changes in the parameters of the breathing waveform; and/or changes in the signature pattern of the breathing waveform. The AI or machine learning algorithm may be configured to detect and/or predict an overdose (e.g. symptoms of an overdose), non-compliance with a set dose or loss of consciousness based on the inputs.

The algorithm may comprise or be comprised in a neural network, a deep neural network, and/or the like.

The AI or machine learning algorithm may be configured to analyse normal breathing waveforms, e.g. for the specific wearer or for a generalized or average wearer, to determine baseline or normal breathing parameters and/or the signature pattern. The AI or machine learning algorithm may be configured to detect changes in the baseline or normal breathing parameters or the signature pattern, e.g. for that specific wearer, to predict or learn how to predict an overdose, non-compliance with a set dose or loss of consciousness. The AI or machine learning algorithm may be configured to learn different overdose detection threshold limits, different predictive breathing waveform parameters, different predictive signature patterns in the breathing waveform, different predictive changes in breathing waveform parameters, and/or different predictive changes in signature patterns in breathing waveforms, for different users of different detectors. The overdose detection threshold limits, different predictive breathing waveform parameters, different predictive signature patterns in the breathing waveform, different predictive changes in breathing waveform parameters, and/or different predictive changes in signature patterns in breathing waveforms may be specific, tailored, bespoke or customized for individual users or wearers. The algorithm may be configured to apply dynamic risk mapping.

The algorithm may be configured to access user data, such as one or more of: age, weight, lifestyle data, risk factors, medical data, history of previous overdoses or non-compliance with a set dose, other medication being taken, other illnesses, breaks in prescription, incarceration history, evidence of variations in drug use, and/or the like. The algorithm may be configured to receive data from other sensors, such as one or more of: heart rate sensors, temperature sensors, and/or the like. The algorithm may be configured to use user data and/or data from other sensors when applying dynamic risk mapping. The algorithm may be configured to use the user data and/or the data from the other sensors in the detecting and/or predicting an overdose, non-compliance with a set dose or loss of consciousness.

The detector and/or the motion sensor may be configured to continuously or periodically monitor motion of the wearer's chest wall. The detector and/or the motion sensor may be configured to determine and/or communicate chest motion of the wearer in real-time or near real time. The detector may optionally be configured to monitor for a set or pre-set, e.g. user input, limited period, e.g. for a period in the order of minutes or hours, such as for 15, 30, 60 or more minutes, or 2, 3, 4 or more hours, or the like.

The processing system, e.g. the AI or machine learning algorithm, may be configured to continuously or periodically predict and/or detect an overdose or non-compliance with a set dose, e.g. in real-time, near real time (i.e. allowing for processing and communication lag and/or discrete digital sampling intervals) and/or on the fly.

The processing system may be configured to detect an overdose or non-compliance with a set dose at least in part by detecting a breathing rate below a threshold limit, such as below a limit of 12 breaths per minute, 8 breaths per minute, or 6 breaths per minute. The processing system may be configured to detect an overdose or non-compliance with a set dose at least in part by detecting a reduction in amplitude of the breathing waveform, e.g. to below a threshold, or by detecting a rate of change in reduction in amplitude of the breathing waveform over time, e.g. by greater than a threshold rate of change. The processing system may be configured to detect an overdose or non-compliance with a set dose at least in part by detecting an absence of the breathing waveform, e.g. indicative of an absence of breathing.

The detector and/or motion sensor may comprise or be configured to connect to a mounting element. The mounting element may be or comprise an adhesive element, which may be configured to adhere to skin. The mounting element may be configured to mount to an article of clothing, such as a vest or bra or on a strap, band or other suitable chest mounting means. The detector and/or motion sensor may be implantable, e.g. implantable in or on the chest, upper abdomen or upper torso.

The mounting element may comprise an electrode, such as a push stud mount electrode.

The mounting element may comprise a contact monitor to monitor contact with the skin. The contact monitor may comprise the electrode. The contact monitor may be a capacitive measurement device, e.g. for measuring a capacitance arising between the measurement device and the skin. The contact monitor may comprise a physical and/or pressure operated contact monitor. The contact monitor may be configured to monitor or determine an electrical parameter, in use, in order to determine whether the detector and/or motion sensor is fixed to the user and/or to determine loss of skin contact. For example, the electrical parameter may comprise impedance such as AC impedance, electrical potential, capacitance, and/or the like. However, any suitable electrical parameter that is indicative of contact or proximity between the electrode and the skin may be used.

The mounting element may comprise a delivery system for delivering a drug or other biologically active material. The mounting element (e.g. delivery system) may comprise an adhesive patch, such as a delivery patch configured to deliver a drug or other medically or biologically active substance. The delivery patch may be an opioid delivery patch. The delivery system may comprise a microneedle array, or other suitable drug delivery system, particularly a trans-dermal drug delivery system. The mounting element may be suitable for mounting the detector for a set period of time, such as a week.

The detector may comprise a power source, such as a battery, inductive charging system, capacitor, wind up electrical generator, kinetic motion generator such as an oscillating weight generator, a heat generator such as a generator that generates electrical current from body heat, a solar or photoelectric generator, and/or the like. The power source may be configured to power the motion sensor, communication system, and/or the detector processing module. The power source may be suitable for powering the detector for a plurality of months, such as six or more months.

The detector and/or the processing system may be configured to provide an alert and/or raise an alarm upon determining or predicting an overdose, non-compliance with a set dose or loss of consciousness. For example, the local processing system (e.g. provided in the digital user device such as a smartphone) may be configured to automatically provide an alert or raise an alarm upon determining or predicting an overdose, non-compliance with a set dose or loss of consciousness. The detector may be configured to automatically control or command the local processing system (e.g. the digital user device) to provide the alert or raise the alarm upon determining or predicting an overdose, non-compliance with a set dose or loss of consciousness. The provision of the alert or raising of the alarm may comprise signalling or messaging a third party, such as a nominated third party, relative, medical professional and/or emergency response service. The provision of the alert or raising of the alarm may comprise generating and/or sending a report, which may comprise at least one of: a device ID, a wearer ID, a location, an indication that an overdose, non-compliance with a set dose or loss of consciousness has been detected or predicted, and/or the breathing rate and/or breathing waveform. The detector and/or the processing system may be configured to provide the alert and/or raise the alarm using the communications system. The alert may comprise an audible alert, a haptic alert, a visual alert and/or the like. The alert may be provided on the digital user device (e.g. the user's smartphone). The alert may comprise the provision of a facility to cancel the alert and a time limit for doing so. The local processing system and/or the remote processing system may be configured to raise an alarm, e.g. with the user's emergency contacts and/or with an emergency service and/or first responder if the alert is not cancelled within the time limit.

The detector and/or processing system may be configured to alter (e.g. reduce or stop, increase or start) delivery of the drug or other biologically active material upon determining or predicting an overdose, non-compliance with a set dose or loss of consciousness. For example, when the detector is mounted using a mounting element in the form of the delivery system for delivering the drug or other biologically active material (e.g. a delivery patch) then the detector may be configured to signal or otherwise control the delivery system to vary (e.g. stop, start, increase or decrease rate of) delivery of the drug or other biologically active material. In this way, the detector may be comprised or for use in a “smart” delivery system for delivering the drug or other biologically active material, e.g. as a safety measure.

The detector and/or one or more components of the detector (e.g. the mounting element and/or a casing for the detector, processor, memory, sensor and/or the like) may be recyclable and/or reusable. The detector and/or the one or more components of the detector may be made from recyclable materials, such as recyclable metals and/or recyclable plastics.

According to a second aspect of the present disclosure there is provided a method of determining and/or predicting a medical condition, such as an overdose, non-compliance with a set dose or loss of consciousness.

The method may comprise determining chest wall movement with a motion sensor, e.g. a chest mounted motion sensor. The method may comprise receiving a signal indicative of chest wall movement from a sensor, such as the chest mounted motion sensor. The method may comprise detecting and/or predicting the overdose or loss of consciousness from the determined chest wall movement. The method may comprise detecting an opioid overdose, non-compliance with a set dose or loss of consciousness.

The method may comprise determining a breathing rate and/or breathing waveform. The method may comprise using the determined chest wall movement to determine the breathing rate and/or breathing waveform. The method may comprise detecting and/or predicting the overdose, non-compliance with a set dose or loss of consciousness from the determined breathing rate and/or breathing waveform.

The method may comprise communicating, such as wirelessly communicating, the detected chest wall movement from the chest mounted motion sensor to a processing system. The processing system may be a processing system as described above in relation to the first aspect. The processing system may comprise at least one of: a detector processing module comprised in the detector; a local processing system that may be at the local location; and/or a remote processing module that may be at the remote location. For example, the local processing system may comprise or be comprised in a digital user device such as a smartphone, personal computer, laptop, tablet computer, phablet, PDA, or other smart device. The remote processing system may comprise or be comprised in a computer, a server system, a workstation, and/or the like. The method may comprise communicating the detected chest wall movement from a chest mounted motion sensor to a remote processing system via the local processing system. The method may comprise communicating between the detector and the local processing system via short or shorter range communications, e.g. via Bluetooth, ZigBee, Wi-Fi, Li-Fi, WiMAX, NFC, and/or the like. The method may comprise communicating with the remote processing system via a network, e.g. a wide area network, cellular phone network or over the internet or via a suitable file or data transfer protocol.

The method may comprise one or more of the following, any of which may be implemented using an algorithm, such as an AI or machine learning algorithm:

using the determined chest wall movement to determine a breathing rate and/or breathing waveform;

using the determined breathing rate or breathing waveform to detect and/or predict an overdose or non-compliance with a set dose (or onset of perceptible overdose symptoms and/or loss of consciousness);

analysing the determined breathing waveform to determine a parameter or change in parameter of the breathing waveform; and

using the parameter or change in parameter of the breathing waveform to detect and/or predict an overdose or non-compliance with a set dose;

detecting the signature pattern in the breathing waveform;

matching the determined signature pattern with a reference signature pattern;

detecting changes in the signature pattern in the breathing waveform, such as a change in amplitude, rate and/or shape of the signature pattern with time; and/or

detecting and/or predict an overdose or non-compliance with a set dose (or onset of perceptible overdose symptoms and/or loss of consciousness) from the signature pattern and/or from changes in the signature pattern detected in the breathing waveform and/or from the matching of the determined signature pattern with the reference signature pattern.

The method may comprise using the AI or machine learning algorithm to learn different overdose detection threshold limits, different predictive breathing waveform parameters, different predictive signature patterns in the breathing waveform, different predictive changes in breathing waveform parameters, and/or different predictive changes in signature patterns in breathing waveforms, for different users, e.g. adapted to or specific for individual users or wearers.

The method may comprise updating the signature patterns with new signature patterns. The method may comprise updating the look-up table of signature patterns with new signature patterns learnt by the AI or machine learning algorithm.

The method may comprise analysing breathing waveform data from multiple users. The method may comprise analysing breathing waveform data from multiple users using an AI or machine learning algorithm. Using breathing waveform data from multiple users may increase the speed with which the AI or machine learning algorithm learns different detection thresholds, different predictive breathing waveform parameters, different predictive signature patterns in the breathing waveform, different predictive changes in breathing waveform parameters, and/or different predictive changes in signature patterns in breathing waveforms, for different users.

The method may comprise accessing user data, such as age, weight, lifestyle data, risk factors, medical data, history of previous overdoses or non-compliance with a set dose, other medication being taken, other illnesses, breaks in prescription, incarceration history, evidence of variations in drug use, and the like. The method may comprise receiving data from other sensors, such as heart rate sensors, temperature sensors, and the like. The method may comprise using user data and/or data from other sensors when applying dynamic risk mapping. The method may comprise using the user data and/or the data from the other sensors in the detecting and/or predicting an overdose, non-compliance with a set dose or loss of consciousness.

The method may comprise continuously or periodically detecting chest wall movement. The method may comprise detecting chest wall movement in real-time. The method may comprise continuously or periodically detecting and/or predicting an overdose or non-compliance with a set dose. The method may comprise detecting and/or predicting an overdose or non-compliance with a set dose in real-time. The method may optionally comprise monitoring for a set or pre-set, e.g. user input, limited period, e.g. for a period in the order of minutes or hours, such as for 15, 30, 60 or more minutes, or 2, 3, 4 or more hours.

The method may comprise communicating breathing waveform data, detected overdoses or non-compliance with a set dose and/or predicted overdoses or non-compliance with a set dose to a medical practitioner. The method may comprise using breathing waveform data, detected overdoses or non-compliance with a set dose and/or predicted overdoses to prescribe opioids, such as dosages and/or timing of opioid administration.

The method may comprise receiving an opioid prescription and/or opioid administration instructions, such as dosage, frequency, and/or timing of opioid administration, from a medical practitioner.

The method may comprise providing a chest mountable overdose detector user with an opportunity and/or mechanism for indicating if a detected overdose or non-compliance with a set dose is a false alarm. The method may comprise being notified of a false alarm of an overdose or non-compliance with a set dose by a user of a chest mountable overdose detector. The method may comprise using the notification of the false alarm to improve the detection of an overdose or non-compliance with a set dose. For example, the method may comprise updating a detection threshold, signature pattern, the algorithm and/or look-up table of signature patterns on the basis that the detected overdose was not an overdose or non-compliance with a set dose.

The method may comprise communicating a predicted overdose and/or a detected overdose to a first responder and/or an emergency responder. This may allow the administration of an antidote, such as naloxone in the case of an opioid overdose.

The method may comprise providing an alert or alarm upon detecting or predicting an overdose or loss of consciousness.

The method may comprise performing any of the operations described above in relation to the first aspect. The method may comprise using the detector of the first aspect.

According to a third aspect of the present disclosure is a processing system configured to perform the method of the second aspect. The processing system may comprise a processor. The processing system may comprise or be configured to communicate with data storage. The processing system may comprise a communications system. The processing system may be, comprise, be comprised in, or distributed over the processing module of the detector of the first aspect, the user device described in relation to the first aspect and/or the remote server described in relation to the first aspect.

The processing system may be configured to receive a signal indicative of chest wall movement obtained using a chest mounted motion sensor; and detecting and/or predicting an overdose and/or loss of consciousness based at least in part on the signal.

According to a fourth aspect is a system comprising the detector of the first aspect and at least one of: a user device and/or a remote processing system. The user device and/or remote processing system may comprise at least one feature of the user device and/or remote processing system described in relation to the first aspect. The user device and/or remote processing system may be configured to perform the method of the second aspect and/or comprise or be comprised in the processing system of the third aspect.

According to a fifth aspect of the present disclosure there is provided a computer program product that, when implemented on a processor such as the processing system described in relation to the first or third aspect of the present disclosure, causes the processing system to implement the method of the second aspect of the present disclosure. The computer program product may be embodied on a non-transient computer readable medium.

The computer program may comprise computer-executable instructions that, when executed by a processor, enable a computer comprising the processor to perform the method of the second aspect of the present disclosure.

The computer program may comprise the AI or machine learning algorithm of the first or second aspect of the present disclosure.

According to a sixth aspect of the present disclosure is a condition detector, which may be an overdose or non-compliance with a set dose detector, such as an opioid overdose detector. The condition detected or predicted by the detector may be or comprise a loss of consciousness, such as a loss of consciousness due to the overdose, e.g. opioid overdose. The detector may be a wearable device, e.g. configured to be worn by a wearer. The detector may be a chest mountable detector. For example, the detector may be configured for mounting to a human chest, such as directly mounting to a human chest. The detector may comprise or be configured to mount to a mounting element. The detector may be configured for mounting directly onto skin. The detector may comprise or be provided on an adhesive mounting element, which may be configured for mounting directly to the skin.

The mounting element may comprise an electrode, such as a push stud mount electrode. The detector may be configured to monitor or determine an electrical parameter using the electrode, in use, in order to determine whether the detector and/or motion sensor is fixed to the user and/or to determine loss of skin contact. For example, the electrical parameter of the electrode may comprise impedance such as AC impedance, electrical potential, capacitance, and/or the like.

The mounting element may comprise an adhesive patch, such as a drug delivery patch or delivery patch for another medical or biologically active compound. The drug delivery patch may be an opioid delivery patch. The detector may comprise and/or be configured to communicate with a processing system. The processing system may be entirely comprised in the detector or at one or more local or remote locations or may be distributed, e.g. functions of the processing system may be provided in the detector and at the local and/or remote locations. For example, the processing system may comprise at least one of: a detector processing module comprised in the detector; a local processing system that may be at the local location; and/or a remote processing system that may be at the remote location. For example, the local processing system may comprise or be comprised in a digital user device such as a smartphone, personal computer, laptop, tablet computer, phablet, PDA, or other smart device. The remote processing system may comprise or be comprised in a computer, a server system, a workstation, and/or the like.

The detector may comprise a communication system. The communication system may be configured to communicate with the local processing system and/or the remote processing system. The communication system may be configured to wirelessly communicate with the processing system. The communication system may be configured to communicate using Bluetooth, ZigBee, WiMAX, Li-Fi, near field communications (NFC) and/or WiFi. The communication system may be configured to communicate with the remote processing system via the local processing system. The communication system may be configured to communicate with the local processing system via short or shorter range communications, e.g. via Bluetooth, ZigBee, Wi-Fi, Li-Fi, WiMAX, NFC, and/or the like. The communication system may be configured to communicate with the remote processing system via a network, e.g. a wide area network, cellular phone network or over the internet.

The processing system may be configured to determine a breathing rate and/or breathing waveform. The processing system may be configured to use the determined chest wall movement to determine the breathing rate and/or breathing waveform. The processing system may be configured to use the determined breathing rate and/or breathing waveform to detect and/or predict an overdose, non-compliance with a set dose or loss of consciousness.

The processing system may be configured to analyse the determined breathing waveform to determine a parameter or change in parameter of the breathing waveform. The processing system may be configured to use the parameter or change in parameter of the breathing waveform to detect and/or predict an overdose, non-compliance with a set dose or loss of consciousness.

The parameter of the breathing waveform may comprise a breathing waveform rate, amplitude, and/or shape of at least part or all of the breathing waveform. The change in parameter of the breathing waveform may comprise a change over time.

The detector and/or processing system may be configured to alter (e.g. reduce or stop, start or increase) delivery of the drug or other biologically active material upon determining or predicting an overdose, non-compliance with a set dose or loss of consciousness. For example, when the detector is mounted using a mounting element in the form of the delivery system for delivering the drug or other biologically active material (e.g. a delivery patch) then the detector may be configured to signal or otherwise control the delivery system to vary delivery of the drug or other biologically active material. In this way, the detector may be comprised or for use in a “smart” delivery system for delivering the drug or other biologically active material, e.g. as a safety measure.

The detector may be or comprise the detector of the first aspect.

According to a seventh aspect of the present disclosure is a system comprising a detector according to the sixth aspect and a user device and/or a remote processing system. The user device and/or a remote processing system may be configured to receive a signal and/or one or more parameters or metrics indicative of movement of a user's chest from the detector.

The user device may be a digital user device such as a smartphone, personal computer, laptop, tablet computer, phablet, PDA, or other smart device. The remote processing system may comprise or be comprised in a computer, a server system, a workstation, and/or the like.

At least one of the user device and/or remote processing system may be configured to implement an algorithm. The algorithm may comprise or be comprised in an artificial intelligence “AI” or machine learning “ML” algorithm. The AI or ML algorithm may be at least partly trained on a known or generated data set and/or on historical or previous data collected by the detector, which may be data for the wearer of the detector. The AI or machine learning algorithm may be configured to receive as inputs one or more of: breathing rate; the breathing waveform; parameters of the breathing waveform such as transitions, maxima and minima or point of inflexion of the breathing waveform, period of the breathing waveform and/or amplitude of the breathing waveform; an indication of presence or absence of the breathing waveform; the signature pattern in the breathing waveform; changes in the parameters of the breathing waveform; and/or changes in the signature pattern of the breathing waveform. The AI or machine learning algorithm may be configured to predict an overdose, non-compliance with a set dose or loss of consciousness based on the inputs.

The algorithm may be distributed over the user device and remote processing system, or separate algorithms may be provided on each of the user device and remote processing system. The algorithm on the remote processing system may be configured to update parameters of the algorithm on the user device, e.g. based on the inputs received by the algorithm. For example, the algorithm on the remote processing system may be configured to update or determine threshold or other trigger conditions for an alarm or alert (such as a number of breaths per minute) and update the algorithm on the user device with the updated or determine threshold or other trigger condition.

The algorithm may comprise or be comprised in a neural network, a deep neural network, and/or the like.

The AI or machine learning algorithm may be configured to analyse normal breathing waveforms, e.g. for the specific wearer or for a generalized or average wearer, to determine baseline or normal breathing parameters and/or the signature pattern. The AI or machine learning algorithm may be configured to detect changes in the baseline or normal breathing parameters or the signature pattern, e.g. for that specific wearer, to learn how to predict an overdose, non-compliance with a set dose or loss of consciousness. The AI or machine learning algorithm may be configured to learn different overdose detection threshold limits, different predictive breathing waveform parameters, different predictive signature patterns in the breathing waveform, different predictive changes in breathing waveform parameters, and/or different predictive changes in signature patterns in breathing waveforms, for different users of different detectors. The algorithm may be user-specific or at least adapted or modified for individual users/wearers.

The algorithm may comprise at least one feature of the algorithm described in relation to the first aspect.

According to an eighth aspect of the present disclosure there is provided a method of determining and/or predicting a medical condition, such as an overdose, non-compliance with a set dose or loss of consciousness. The method may comprise providing a detector, which may be an overdose detector or detector for detecting non-compliance with a set dose, such as an opioid overdose detector. The method may comprise mounting the device to a user. The method may comprise mounting the detector to a chest of a user, such as directly mounting to a human chest. The method may comprise mounting the detector to the user using a mounting element. The mounting element may comprise a push stud mount electrode. The mounting element may comprise an adhesive patch, such as a drug delivery patch. The drug delivery patch may be an opioid delivery patch.

The method may comprise detecting chest wall movement with the detector, e.g. a chest mounted motion sensor. The method may comprise detecting and/or predicting the overdose from the chest wall movement. The method may comprise detecting an opioid overdose or loss of consciousness.

The method may comprise determining a breathing rate and/or breathing waveform. The method may comprise using the detected chest wall movement to determine the breathing rate and/or breathing waveform. The method may comprise detecting and/or predicting the overdose from the determined breathing rate and/or breathing waveform.

The method may comprise performing the method of the second aspect.

It should be understood that the individual features and/or combinations of features defined above in accordance with any aspect of the present disclosure or below in relation to any specific embodiment of the disclosure may be utilised, either separately and individually, alone or in combination with any other defined feature, in any other aspect or embodiment of the disclosure.

Furthermore, the present disclosure is intended to cover apparatus configured to perform any feature described herein in relation to a method and/or a method of using or producing, using or manufacturing any apparatus feature described herein.

### BRIEF DESCRIPTION OF THE DRAWINGS

At least one embodiment of the disclosure will now be described, by way of example only, with reference to the accompanying drawings, in which:

FIG. 1 shows a system comprising a chest mountable overdose detector, a local computer and a remote computer;

FIG. 2 illustrates the system of FIG. 1 in use; and

FIG. 3 illustrates a method of use of the system of FIG. 1

### DETAILED DESCRIPTION OF THE DRAWINGS

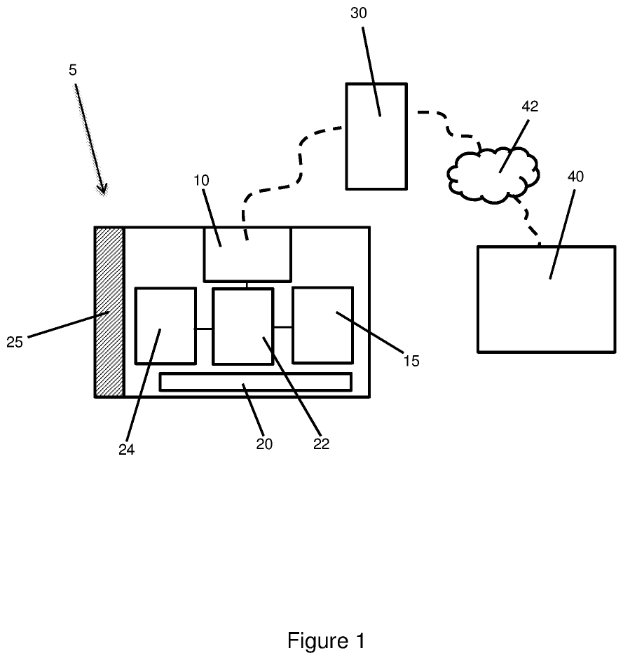

FIG. 1 shows a chest mountable detector 5, which in this example is a detector for detecting an opioid overdose. The chest mountable detector 5 comprises a communication system 10, a motion sensor 15 that in this example is an accelerometer, and a battery 20. Optionally (but not essentially), the detector 5 may also comprise a processing module 22 and/or data storage 24. The detector 5 comprises, is comprised in or on or is mountable to a delivery patch 25, which in this embodiment is an opioid delivery patch. However, in other embodiments, the detector 5 can be mounted via other mounting means, such as an adhesive skin electrode (such as a popper stud electrode used in the collection of ECG measurements and other applications). The communication system 10, the accelerometer 15, the data storage 24 are in data communication with the processing module 22 and are powered by the battery 20.

The communication system 10 is configured to communicate wirelessly with a user device 30, such as a smartphone. An app on the user device is configured to provide a user interface and appropriate communications protocols for communicating with the detector 5. The user device is configured to communicate wirelessly with a remote server 40, e.g. over the internet 42 via the cellular communication or Wi-Fi capabilities of the user device 30. In an embodiment, the remote server 40 implements an AI algorithm that is configured to analyse the chest motion detected by the accelerometer. However, in other embodiments, it will be appreciated that the algorithm could be implemented by the user device 30 and/or by the processing module 22 on the detector 5 or distributed between any or all of the processing module 22, the user device 30 and the remote server 40.

The detector 5 is configured to be mounted on the upper torso or upper abdomen of a user (e.g. on or around the chest) using the patch 25. The patch 25 is generally provided with an adhesive layer suitable for adhering the patch 25 to skin. The patch 25 contains a drug or other medically active substance, in this case an opioid, which is configured to be effused from the patch 25 in a controlled manner over a controlled timeframe to be absorbed by the user via the skin. The other components of the detector 5, e.g. the communication system 10, motion sensor, battery 20, and processing module 22, can be comprised in or on the patch 25 or mounted or releasably mountable onto the patch 25 using any suitable means. In one possible example, a backing layer of the patch 25 comprises a polymeric film that can be conveniently used as a substrate upon which the other components of the detector 5 can be provided, but the present disclosure is not limited to this. By incorporating the detector 5 into the delivery patch 25 that is used to deliver a drug or other medically active substance (in this case an opioid), the user is less likely to remove or forget the detector 5.

As the chest wall of the user moves as the user breathes the detector 5 mounted on the upper torso or upper abdomen of the user also moves correspondingly. The motion sensor 15 provides an output signal that depends on the motion of the detector 5 and thereby the motion of the chest wall of the user, in use.

The processing module 22 may comprise a logic circuit, processor, integrated circuit (IC), field programmable gate array (FPGA), application specific integrated circuit (ASIC) or any other suitable digital processing means. The processing module 22 is arranged to receive the output signal from the motion sensor 15. In embodiments, the processing module 22 can be configured to process the output signal 15 to derive parameters or metrics (e.g. breathing rate amplitude of chest wall motion and/or breathing waveform or properties thereof such as period, amplitude, points of inflexion, transitions, presence or absence of the breathing waveform, and/or the like) therefrom or in other embodiments the processing module effectively does no more than condition the signal from the motion sensor 15 so as to be suitable for communication by the communications system 10. In other embodiments, the motion sensor 15 provides the output signal directly to the communications system 10. Optionally, data indicative of the sensor signal (or the parameters or metrics derived therefrom) can be temporarily stored in the data storage, but preferably they are continuously communicated to the user device 30 using the communications system 10 in real time or near real time (e.g. accounting for processing and communications lag and/or discrete digital sampling periods).

The signal that is representative of the motion of the chest wall (or the parameters or metrics derived therefrom) is processed by the algorithm. It will be appreciated that the determination of the parameters or metrics (such as breathing rate, presence, absence or amplitude of chest wall motion and/or breathing waveforms or properties thereof such as period, amplitude, points of inflexion, transitions and/or the like) may be performed by or distributed on any of the processing module 22, the user device 30 and/or the remote server 40. In some embodiments, as little processing as possible is performed by the detector 5 and all or at least most of the processing is performed on either the user device 30 or the remote server 40. In this way, the detector 5 can be made very cheaply and is effectively disposable. In some embodiments, the detector 5 is processor-less, i.e. no processing module is provided or the processing module 22 does not contain a processor and simply provides only means sufficient to condition the signal output by the motion sensor for communication by the communications system 10 and no more. However, in implementations where power consumption, reducing and delays in raising the alarm, communications bandwidth and/or security are deemed more important, then the processing module 22 may be configured to perform signal processing such as signal or data compression, encryption, extraction of the parameters or metrics, and/or the like. It will be appreciated that different applications may require different trade-offs and the processing performed and the device carrying out that processing, whether that is on the processing module 22 of the detector 5, on the user device 30, the remote server 40, or any combination of those or even switchable between them, may be selected depending on the particular application.

The algorithm is adapted to analyse the signal that is representative of the motion of the chest wall or preferably the parameters or metrics such as presence or absence of breathing, breathing rate, amplitude of chest motion and/or breathing waveform, in order to determine if the user has taken an overdose or is experiencing or likely to soon experience a loss of consciousness. Factors that may be identified by the algorithm include a reduction in breathing rate, a reduction in amplitude range of the breathing waveform, and/or the like. An overdose or loss of consciousness may be identified or predicted by comparing these parameters or metrics to thresholds or other trigger criteria (which may be dynamically determined based on recent data for that user) or may be determined by comparison of a rate of change in these parameters to see if it is over a threshold, and/or the like. The thresholds or other trigger thresholds may be adapted for or specific to individual users/wearers, and may be set using an algorithm, such as an AI or machine learning algorithm, which may be implemented on the processing module 22 of the detector 5, on the user device 30 and/or on the remote server 40.

A more advanced method is to use an artificial intelligence (AI) or machine learning (ML) algorithm, which may be trained on historical data (e.g. comprising the signal from the motion sensor 15 that is representative of motion of the chest wall and/or the parameters or metrics derived therefrom) for the user and/or training set data. The AI or ML algorithm may be configured to learn from the historical and/or training data to identify “normal” or baseline data, pre-emptive data that is indicative of a high probability that overdose symptoms or loss of consciousness will likely soon occur (e.g. after an overdose has been administered but before the full extent of symptoms that would be recognised by the user, such as loss of consciousness, become apparent) or occurrence data that is indicative of full overdose symptoms or loss of consciousness.

The algorithm can take into account more parameters than simply those received form the detector 5. For example, the remote server 40 or the app running on the user device 30 may be configured to access user data such as age, weight, lifestyle data, risk factors, medical data, history of previous overdoses or non-compliance with a set dose, other medication being taken, other illnesses, breaks in prescription, incarceration history, evidence of variations in drug use, and the like. Furthermore, the remote server 40 or the app running on the user device 30 may be configured to receive data from other sensors, such as heart rate sensors, temperature sensors, and the like. The user data and/or the data from other sensors can also be fed into the algorithm, which may improve the efficacy of the algorithm in detecting and/or predicting overdoses and/or loss of consciousness of the user.

Once the algorithm has identified that an overdose or loss of consciousness has occurred or that overdose symptoms or loss of consciousness are likely to imminently occur, the algorithm triggers an alert procedure. The alert procedure may be customizable by the user via the app running on the user device 30 and/or remotely by a remote user such as a medical professional or alert service provider, e.g. via the remote server 40. In this way, the alert settings can be remotely supervised, set and/or monitored.

The alert procedure may be provided by the detector 5, or preferably by the user device 30 and/or by the server 40. For example, the alert procedure could be configured to provide one or more of a visual alert such as a flashing light, notification or the like, or an audible alert or a haptic alert such as a vibration alert. The alert procedure may comprise notifying (e.g. calling or messaging) an emergency contact, emergency service and/or first responder and providing assisting data such as a location, identify of the user, an indication of the determined condition, the parameters or metrics and any changes/patterns therein, etc. For example, the alert procedure may comprise providing a visual, audible and/or haptic alert to the user, e.g. using the user device 30. Unless the user cancels the alert within a set or default time period, then the user device 30 is configured to provide the alert notification to the emergency contact, emergency service and/or first responder.

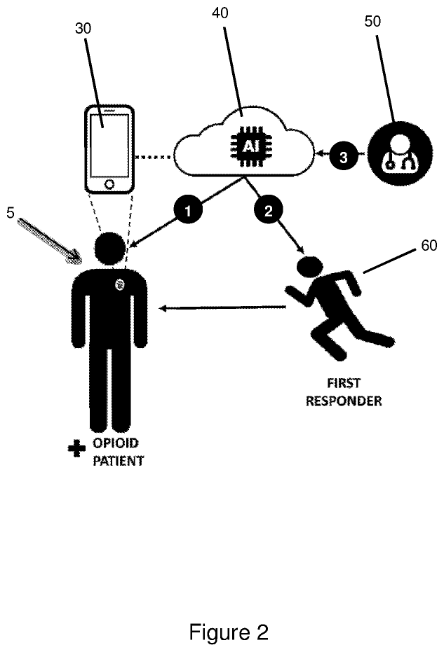

FIG. 2 illustrates a use of the chest mountable overdose detector 5 of FIG. 1. The chest mountable overdose detector 5 and the user device (e.g. smartphone) 30 communicate wirelessly with one another, e.g. using Bluetooth, Zigbee, Wi-Fi, Li-Fi, WiMAX, NFC or some other suitable local wireless communications protocol. The smartphone 30 is configured to communicate with the cloud based AI algorithm provided by the remote server 40, e.g. using a cellular telephone network and/or Wi-Fi. The cloud based AI algorithm and the smart phone 30 both perform real-time (or near real time, e.g. accounting for processing and communication lag and/or discrete sampling periods, which may be in the order of seconds or minutes, e.g. 5, 15, 30 or 60 second sampling periods) breathing waveform analysis using the continuous motion detected by the chest mountable overdose detector 5. The AI algorithm in the cloud 40 performs a real-time or near real-time analysis of the breathing waveform of each user to map the risk of overdose and predict an overdose. Optionally, the analysis performed by the algorithm 40 can be extended to automatically suggest variations in the administration of the drug, such as variations in timing, frequency and/or dosing of a drug (e.g. of the opioid). This may comprise automatically recommending or adjusting dose amounts and/or timing. The recommendation may be provided to the user/wearer in order to get them to manually adjust the administration of the drug, e.g. by adjusting timing, frequency and/or dosing of the drug, or may be automatically provided in the form of a control command to a delivery system (e.g. a “smart patch” 25) that causes the delivery system to automatically adjust the administration of the drug, e.g. by adjusting timing, frequency and/or dosing of the drug, and/or may be provided to the user's medical practitioner to implement, as further explained below.

The analysis performed by the AI algorithm in the cloud 40 is available to a medical practitioner 50. The medical practitioner 50 can use the analysis from the AI algorithm to change an opioid prescription and/or opioid administration instructions. For example, the medical practitioner 50 may increase an opioid dose if the opioids are not having sufficient effect, or decrease an opioid dose if there is a frequent risk of overdose, or change the timing, frequency and/or frequency of the opioid administration as required. In the event of an automatic variation of the administration (e.g. timing, frequency and/or dosing) or of an instruction to the user/wearer to manually vary the administration (e.g. the timing, frequency and/or dosing), then the medical practitioner 50 can be automatically alerted or an appropriate entry logged in a database on in the user/wearer's medical record.

The new dosage and timing instruction are sent 1 to the user of the chest mountable overdose detector 5 from the medical practitioner 50 via the AI algorithm in the cloud 40. If an overdose is detected, the AI algorithm in the cloud 40 contacts 2 a first responder 60 to aid the user of the chest mountable overdose detector 5, and administer an opioid overdose antidote, if necessary. In response to the overdose and the subsequent alert from the cloud based algorithm 40, a medical practitioner 50 sends updated dosage and timing instructions 3 to the AI algorithm for sending to the user of the chest mountable overdose detector 5.

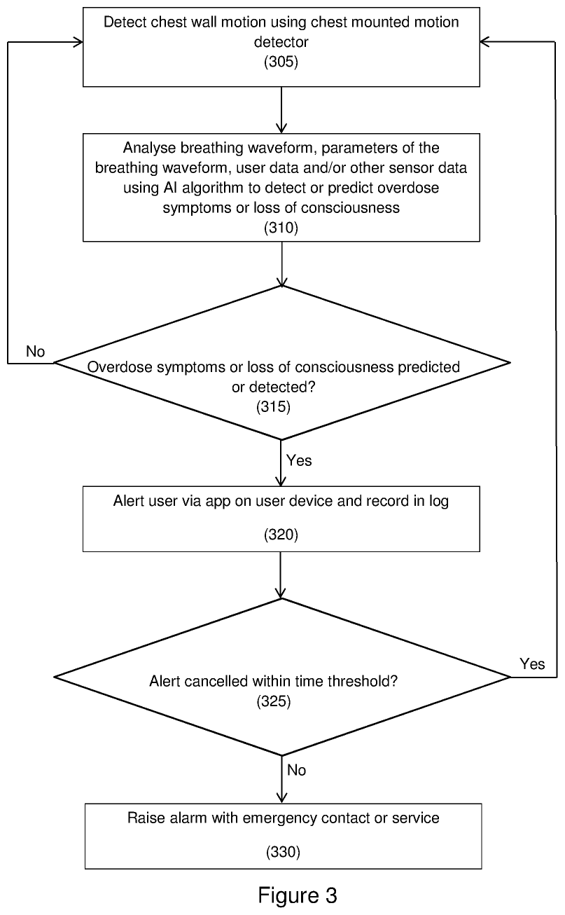

FIG. 3 illustrates a method of detecting an overdose or loss of consciousness using the detector 5 of FIGS. 1 and 2. It will be appreciated that the steps of the method illustrated in FIG. 3 may be carried out by a suitable processing system operating under instructions comprised in a computer program product. The processing system may comprise one or more of: the processing module 22, the user device 30 and/or the remote server 40. It will be appreciated that the detector 15 is configured to communicate with the user device 30 via the communications system 10 using short range communications such as Bluetooth and Wi-Fi and the user device 30 is in turn configured to communicate with the remote server 40 over a cellular communications network or over Wi-Fi via the internet or via a suitable file or data transfer protocol. As such, the method may be performed by any of, or distributed over one or more of, or selectively switched between, the detector 15, user device 30 and/or remote server 40 depending on considerations such as detector cost, size and power consumption, network bandwidth, latency, network availability and/or the like.

In step 305, the output signal from the motion sensor 15 that is representative of chest wall motion of the user is received in real time or near real time and analysed in real time or near real time to determine parameters and metrics of the user's breathing, such as presence or absence of breathing, breathing rate, breathing waveform and the range or amplitude of chest wall motion. Other data such as the user data (e.g. user age, weight, lifestyle, medical data, history of drug use, etc.) retrieved from a data base and/or other sensor data received from other sensors such as heartrate of the user, temperature, etc. may be received.

In step 310, the parameters and metrics and optionally also the other data may be analysed using an AI or ML algorithm that has optionally been trained on a training data set and/or using historical data derived from the motion sensor 15 for the user and/or the other data. The AI or ML algorithm is configured to return an indication that the breathing of the user is normal, or that imminent overdose symptoms or loss of consciousness are predicted with a likelihood higher than a set or pre-set threshold, or that overdose symptoms or loss of consciousness are currently detected (step 315).

If the determination in step 315 is that no overdose symptoms or loss of consciousness is predicted or detected, then the process returns to step 305 and the output of the motion sensor 15 continues to be monitored in real time or near real time. If the determination in step 315 is that overdose symptoms or loss of consciousness symptoms are predicted or detected (e.g. overdose symptoms that would be obvious to the user are predicted to occur or are presently detected), then an alert is provided to the user, preferably using the user device 30 (e.g. smartphone). The alert may be in the form of a visual, audible and/or haptic alert, for example. The detection or prediction is also recorded in a log in a database (e.g. on the user device 30 and/or on the remote server 40). This information can be used to feed back to the user's health care professional and/or used to produce training data for training the AI or ML algorithm. The alert comes with an option for the user to cancel the alert, e.g. by pressing a button or virtual button on the user device 30 or by using a voice command.

If the alert is cancelled within a set or pre-set time limit, then the rest is logged (e.g. to produce training data for the algorithm and to feedback to the user's medical professional) and, with an optional delay in order to avoid continuous re-triggering, the process reverts back to step 305 and continues to monitor the output signal from the motion sensor 15.

However, if the alert is not cancelled by the user within the time limit, then an alarm is raised, e.g. by the app running on the user device 30 or the remote server contacting emergency contacts whose details were previously provided and stored and/or by contacting emergency service or a first responder. The alarm may include further data to assist the emergency contact or emergency service, such as the location of the user (e.g. obtained using the satellite positioning system of the user device 30), a classification of the prediction or detection made by the algorithm, the identity or other details of the user and/or the like. The non-cancelled alert is also logged in the database (e.g. to produce training data for the algorithm and to feedback to the user's medical professional).

The database may be maintained by or accessible from the remote server 40 and can be accessed by medical professionals in order to identify patterns of behaviour, such as misuse. The remote server 40 can optionally be configured to identify patterns or trends in the logged data (e.g. determined overdoses and determined or predicted loss of consciousness or in the raw breathing data or waveforms or in the metrics and parameters derived therefrom). The remote server 40 can then automatically notify the user's medical professional based on set or pre-set patterns of behaviour (e.g. overdoses) detected by the system and logged in the database. In this way, it can be easier for medical professions to identify potentially problematic behaviour and act accordingly, e.g. by adjusting doses, changing prescriptions or taking other preventative or prophylactic actions.

The processing module 22 of the detector 5, the local processing system 30 and/or the remote server 40 may be configured to alter (e.g. reduce or stop, start or increase) delivery of a drug or other biologically active material upon determining or predicting an overdose, non-compliance with a set dose or loss of consciousness. For example, when the detector 5 is with a delivery system for delivering the drug or other biologically active material (e.g. the delivery patch 25 or a microneedle array) then the detector 5 can be configured to signal or otherwise control the delivery system to vary delivery of the drug or other biologically active material. For example, if the delivery system is configured to deliver an opioid, then the detector may be configured to automatically control or signal the delivery system to reduce or stop delivery of the opioid. In another example, if the delivery system is configured to deliver a treatment drug, such as an asthma drug or a drug to be taken in the event of anaphylaxis or allergic reaction, then the detector may be configured to automatically control or signal the delivery system to start or increase delivery of the treatment drug. In these embodiments, the detector can be considered comprised or for use in a “smart” delivery system for automatically delivering the drug or other biologically active material, e.g. as a safety measure.

The above examples are provided by way of illustration only and a skilled person would appreciate that modifications to the above examples could be made. For example, although the above example uses an AI algorithm in the cloud, one skilled in the art will understand that the breathing waveform analysis may be performed by any suitable computer, local to the chest mountable overdose detector or in a remote location.

In addition, although an example of use of an AI or ML algorithm that directly determines whether an overdose has been taken and/or whether loss of consciousness has occurred or is imminent or likely, the algorithm may operate in different manners. For example, the determination of whether an overdose has been taken and/or whether loss of consciousness has occurred or is imminent or likely may be carried out on the user device 30 and/or on the processing module 22 on the detector 5 using a method that comprises comparing one or more metrics or parameters of the breathing or breathing waveform, such as breathing rate or amplitude or period, amplitude, points of inflexion or transitions in the breathing waveform, with one or more thresholds or other trigger conditions to determine wither an alert or alarm should be raised/sent. The algorithm on the remote server 40 (and/or user device 30), e.g. the AI or ML algorithm can be configured to update or adjust the one or more thresholds or other trigger conditions, e.g. based on breathing waveform data (e.g. historical data) collected by the detector 5, the user data and/or the data from other sensors, which may comprise user specific data and/or aggregated data.

Although examples are given above in which the detector 5 is mounted to a chest, upper abdomen or upper torso of a user/wearer using a drug delivery patch or adhesive electrode, it will be appreciated that other arrangements for mounting the detector 5 could be used. For example, the detector could be provided on any suitable adhesive patch or pad, in an article of clothing that is worn around the chest, upper abdomen or upper torso such as in a vest or bra, on a chest strap or belt or implanted on or in the user/wearer.

References to prediction of overdose symptoms may mean detection of parameters or metrics that imply that an overdose is likely to have been administered but that the full symptoms have not yet been reached (e.g. the user hasn't yet lost consciousness) and/or will be apparent to the user. References to detection of overdose symptoms may mean a determination that the overdose symptoms have progressed to an advanced level and/or have reached the stage that they will be readily apparent to the user, e.g. resulting in a loss of consciousness or significantly reduced consciousness.

Although the detector and associated systems and methods described above are particularly beneficial in detecting overdoses, particularly opioid overdoses, it will be appreciated that they may be used instead to detect other conditions, particularly those that may ultimately result in a loss of consciousness, and so the present disclosure is not so limited.

Furthermore, although certain approaches are described above, these are provided for understanding only and it will be appreciated that other approaches may be used instead. For example, if a first responder is not suitably close to the user of the chest mountable overdose detector to provide aid quickly enough, a central help centre or emergency responder may be contacted instead.

As such, the scope of disclosure is not limited by the above examples but only by the claims.

1. A condition detector, the condition detector comprising:
a motion sensor mounted or mountable to a chest wall, upper abdomen or upper torso, wherein the motion sensor is configured to determine chest wall movement, and
the condition detector is configured to detect and/or predict drug dose non-compliance, based on the chest wall movement determined by the motion sensor.

2. The condition detector of claim 1, wherein the non-compliance is an opioid overdose.

3. The condition detector of claim 1, wherein the motion sensor is an accelerometer.

4. The condition detector of claim 1, wherein
the condition detector is configured to use the detected chest wall movement to determine a presence or absence of breathing, breathing rate, amplitude of chest wall motion associated with breathing, and/or breathing waveform; and
the condition detector is configured to analyse the presence or absence of breathing, breathing rate and/or breathing waveform to detect and/or predict the overdose or other drug dose non-compliance.

5. The condition detector of claim 1, wherein the motion sensor is provided or mountable on a mounting element, wherein the mounting element consists of one or more of: a delivery system for delivering a drug or biologically active material, an electrode system, a patch, an adhesive mounting system and/or a mount for mounting the motion sensor to an article of clothing.

6. The condition detector of claim 5, wherein the mounting element is configured to deliver an opioid.

7. The condition detector according to claim 5, wherein the mounting element further comprises a contact monitor to monitor contact with the skin.

8. The condition detector of claim 1, further comprising a communication system, wherein the communication system is configured to communicate with a local processing system and/or with a remote processing system; and at least one of a local processing system and/or a remote processing system, wherein the local processing system and/or a remote processing system is configured to:
receive at least one of: a signal, a parameter and/or a metric indicative of chest wall movement of a wearer of the condition detector;
use the chest wall movement to determine a breathing rate or breathing waveform; and at least one of:
use a determined breathing rate or breathing waveform to detect drug dose non-compliance, and/or
use a determined breathing rate or breathing waveform to detect and/or predict an overdose or other drug dose non-compliance, overdose symptoms and/or loss of consciousness.

9. The condition detector of claim 8, wherein the local processing system and/or a remote processing system is configured to:
analyse the determined breathing waveform to determine a breathing waveform rate, amplitude, shape, and/or changes in any parameters of the breathing waveform with time; and
use the determined breathing waveform rate, amplitude, shape, and/or determined changes in any parameters of the breathing waveform with time, to detect and/or predict an overdose or other drug dose non-compliance.

10. The condition detector of claim 8, wherein the local processing system and/or a remote processing system is configured to:
detect a signature pattern in the breathing waveform;
match the signature pattern to a reference signature pattern;
detect changes in the signature pattern in the breathing waveform; and
detect and/or predict an overdose or other drug dose non-compliance from the signature pattern and/or from changes in the signature pattern detected in the breathing waveform and/or matched to the breathing waveform.

11. The condition detector of claim 8, wherein the local processing system and/or a remote processing system is configured to apply filtering to the determined breathing waveform to filter out determined motions that have a frequency higher than an upper threshold and/or lower than a lower threshold.

12. The condition detector of claim 9, wherein the detector and/or the local processing system is configured to determine whether an overdose has been taken and/or whether loss of consciousness has occurred or is imminent or likely using a method that comprises comparing one or more metrics or parameters of the breathing or breathing waveform with one or more thresholds or other trigger conditions to determine wither an alert or alarm should be raised/sent and local processing system and/or a remote processing system configured to update or adjust the one or more thresholds or other trigger conditions.

13. A method of determining and/or predicting a medical condition, the method comprising
detecting chest wall movement with a chest mounted motion sensor; and
detecting and/or predicting the medical condition from the chest wall movement.

14. The method of claim 13, wherein the detected condition is an opioid overdose or other drug dose non-compliance.

15. The method of claim 13, the method comprising:
using the detected chest wall movement to determine a breathing rate or breathing waveform; and
analysing the determined breathing rate or breathing waveform to detect and/or predict the condition.

16. At least one non-transitory computer readable storage medium for determining and/or predicting a medical condition, the at least one non-transitory computer readable storage medium storing computer executable instructions that, when loaded into computer memory and executed by a processor cause the processor to perform the following steps:
using the detected chest wall movement to determine a breathing rate or breathing waveform; and
analysing the determined breathing rate or breathing waveform to detect and/or predict the condition.

17. A chest mountable detector for detecting a medical condition, the detector being configured for mounting to the skin of a wearer, wherein the detector comprises or is configured to mount to a mounting element that is skin mounted or mountable, and the mounting element comprises an adhesive electrode or a drug or other biologically active compound delivery patch.

18. The detector of claim 17, wherein the detector is an opioid overdose detector and the drug delivery patch is an opioid delivery patch, and the detector comprises a motion sensor for monitoring motion of the chest upon which it is mounted.

19. A system comprising:
a detector being configured for mounting to the skin of a wearer, wherein the detector comprises or is configured to mount to a mounting element that is skin mounted or mountable, and the mounting element comprises an adhesive electrode or a drug or other biologically active compound delivery patch and at least one of: a local processing system in the form or a digital user device and a remote processing system, wherein the detector comprises a wireless communications system for communicating data from the motion sensor to the local processing system or the remote processing system, wherein the local processing system or the remote processing system are configured to:
determine or receive a breathing rate and/or breathing waveform using the chest wall motion determined using the motion sensor; and
use the determined breathing rate and/or breathing waveform to detect and/or predict the medical condition.

20. A method of determining and/or predicting a medical condition, the method comprising:
providing a detector being configured for mounting to the skin of a wearer, wherein the detector comprises or is configured to mount to a mounting element that is skin mounted or mountable, and the mounting element comprises an adhesive electrode or a drug or other biologically active compound delivery patch;
mounting the detector to the skin of a user using the mounting element of the detector;
detecting chest wall movement with a chest mounted motion sensor; and
detecting and/or predicting the condition from the chest wall movement.
